(19) United States
(12) Patent Application Publication
(10) Pub. No.: US 2018/0008570 A1
(43) Pub. Date: Jan. 11, 2018

(54) METHOD OF TREATMENT OF A NEUROPATHIC PAIN

(71) Applicant: CHAOYANG UNIVERSITY OF TECHNOLOGY, Taichung City (TW)

(72) Inventors: Meng-Jen Lee, Nantou City (TW); Hsin-Sheng Tsay, Taichung City (TW); Huang-Chi Wang, Kaohsiung City (TW)

(73) Assignee: CHAOYANG UNIVERSITY OF TECHNOLOGY

(21) Appl. No.: 15/223,350

(22) Filed: Jul. 29, 2016

(30) Foreign Application Priority Data
Jul. 7, 2016 (TW) 105121616

(51) Int. Cl. A61K 31/341; A61K 9/00

(52) U.S. Cl. CPC A61K 31/341; A61K 9/0019

(57) ABSTRACT

A method of treatment of a neuropathic pain by administering an andrographolide to the subject is provided. The andrographolide treatment may relief the symptom of neuropathic pain by reducing the astrocytic activity.

### CROSS-REFERENCE TO RELATED APPLICATION

This application claims priority from Taiwan Patent Application No. 105121616, filed on Jul. 7, 2016, in the Taiwan Intellectual Property Office, the content of which is hereby incorporated by reference in their entirety for all purposes.

### BACKGROUND OF THE INVENTION

### 1. Field of the Invention

The present invention relates to a method of treatment of a neuropathic pain, and more particularly to a method of treatment of a neuropathic pain by administering an andrographolide to the subject to relief the symptom of the neuropathic pain.

### 2. Description of the Related Art

Andrographis paniculata Nees (Family Acanthaceae) is a common Chinese medicament primarily used for its effects of detoxification, blood cooling and swelling relief. It is generally used for the treatment of acute dysentery, gastroenteritis, the common cold, bronchitis, whooping cough, tuberculosis, lung abscess, cholecystitis, hypertension, epistaxis, oropharyngeal swelling, bolis carbuncle, thermal burns, snakebite, as well as other conditions or afflictions. A large quantity of Andrographis paniculata Nees is cultivated in tropical Asia.

Andrographis paniculata Nees extract contains a large quantity of compounds including flavonoids and labdane diterpene. Botanical extracts from Andrographis paniculata Nees have been reported which have anti-filarial, antimalarial, antibacterial, antimicrobial, liver protecting, 2E1 (aniline hydroxylase) suppressing, antihypertensive, anticancer, antipyretic, anti-inflammatory, anti-diabetic, and other beneficial properties. Extract from Andrographis paniculata Nees also has a sedative effect and increases the duration of sleep in mice.

Andrographolide is a diterpenoid which is capable of protecting mice's liver cells from damage caused by paracetamol, reducing damages to the mice's liver caused by carbon tetrachloride (CTC) or tBHP, and preventing platelet aggregation. In addition, andrographolide also seems capable of increasing the cell number of CD⁴⁺ lymphocytes to inhibit the activity of the human immunodeficiency virus (HIV), inhibiting the growth and spread of various cancer cells, and reducing inflammation. Andrographolide leads to lower glucose levels in a diabetic mouse, and to suppress cell apoptosis in human endothelial cells which lack of growth factors. Andrographolide also has been report which may moderate the immune response, preventing hepatic toxicity, preventing oxidation, and also protecting the cardiovascular system, including the heart.

Neuropathic pain is a pain aroused and caused by a primary lesion or dysfunction of the nervous system, and the source of the neuropathic pain is mainly divided into peripheral nervous or central nervous according to the position of the primary lesion or dysfunction in the nerves. In 2011, the definition of neuropathic pain is narrowed down to specifically a pain directly caused by lesions or disease of somatosensory nervous system, and leading to abnormal sensations such as dysesthesia, or allodynia. The patient, who is suffering from neuropathic pain, becomes very sensitive to external stimulations and even feels pain from normally non-painful stimuli.

Treatments for neuropathic pain are mostly with opioids, steroids or non-steroidal anti-inflammatory drugs (NSAIDs). These, however, give no pain relief in approximately 10% of patients. Use of opioids is suitable for short-term use, but there is a risk of addiction in long-term use. The class of steroids, such as glucocorticoids, acts by binding to the glucocorticoid receptor (GR), a nuclear receptor, and then the glucocorticoids translocate itself into the cell nucleus and binds with several promoter regions of anti-inflammatory genes. It further interferes with the transcription factor NF-κB, preventing it from binding with DNA in the promoter region. However, its effect is not specific, and long-term use may give rise to immunodeficiency, growth retardation, hypertension, osteoporosis, and other undesirable side effects. NSAIDs act by inhibiting the activity of cyclooxygenase-1 (COX-1) and cyclooxygenase-2 (COX-2) isoenzymes and so inhibiting the synthesis of prostaglandins and thromboxane, which are necessary in the process of inflammation. It is thought that the specific inhibition of COX-2 leads to the anti-inflammatory, analgesic and antipyretic effects. However, most NSAIDs also inhibit COX-1, and these NSAIDs (such as aspirin) may cause gastrointestinal bleeding and ulcers. These are the conventional treatments used to treat neuropathic pain and, as just described, are in general not suitable for long-term use.

Up-regulation of immunoreacitvity for GFAP was often regarded as an indicator of astrocytic reactivity. Hypertrophy of astrocytes demonstrated by up-regulation of GFAP was associated with pain states in several trauma pathology. In CNS inflammation state the astrocytic reaction could outlast the microglial reactivity, and astrocytic reaction could be induced independently to microglia activation. Activated astrocytes alone are sufficient to induce pain sensation, for example, in the case of stem cell implantation into spinal cord. The astrocytes is uniquely rich in gap junction (especially CX30 and CX40), of those the level were increased after central injury. Inhibition of gap junction was demonstrated to be anagelsic, especially for mirror pain transduction.

Following extensive research, which included carrying out several experiments, the inventor of the present invention provides a method of treatment of a neuropathic pain with the aim of addressing the aforementioned limitations of the prior art.

### SUMMARY OF THE INVENTION

The primary objective of the present invention is to overcome the aforementioned limitations of the prior art in the treatment of neuropathic pain.

To that aim, herein disclosed is a method of treatment of a neuropathic pain in a subject, comprising administering an andrographolide to the subject to relief the symptom of the neuropathic pain by reducing the activity of astrocytes.

Preferably, the reducing of the astrocytic activity includes reducing the cellular level of glial fibrillary acidic protein (GFAP).

Preferably, the neuropathic pain is a central nervous system pain.

Preferably, the andrographolide is administered to the subject in a therapeutically effective amount.

Preferably, the causes of the neuropathic pain include spinal cord trauma, a cerebrovascular disease, multiple sclerosis, a tumor, diabetic peripheral neuropathy, a herpes zoster infection, HIV-AIDS, a chronic injury, an acute injury or an autoimmune disorder.

Preferably, the andrographolide is administered orally, intraperitoneally, intravenously, intramuscularly, or subcutaneously to the subject.

Preferably, a medicament containing the andrographolide includes an acceptable carrier, a diluent, or an excipient.

The present invention is further described and illustrated in the preferred embodiment and accompanying drawings that follow. The technical characteristics, implementation and advantages of the present invention are explained in more detail therein.

### BRIEF DESCRIPTION OF THE DRAWINGS

FIG. 1 is a histogram showing the mice's right hindpaw withdrawal threshold (in grams) in a Von Frey test. OP-AP group withdrawal thresholds are significantly increased compared to OP-saline group (*) and OP-NSAIDs group (#) respectively. * or #: P<0.05, * * or ##: P<0.01, * * * or ###: P<0.005.

FIG. 2 is a histogram showing the mice's left hindpaw withdrawal threshold (in grams) in a Von Frey test. OP-AP group withdrawal thresholds are significantly increased compared to OP-saline group (*) and OP-NSAIDs group (#) respectively * or #: P<0.05, * * or ##: P<0.01, * * * or ###: P<0.005.

FIG. 3 is a histogram showing the withdrawal rates of reaction types II˜IV of a fiber of 0.02 g in a Von Frey test. Withdrawal rate is determined by (number of withdrawal)/10. Lower withdrawal rate indicates less mechanical allodynia. OP-AP group is analyzed against OP-saline group (*) and OP-NSAID group (#). * or #: P<0.05, * * or ##: P<0.01, * * * or ###: P<0.005.

FIG. 4 is a histogram showing the withdrawal rates of reaction types II˜IV of a fiber of 0.07 g in a Von Frey test. Withdrawal rate is determined by (number of withdrawal)/10. Lower withdrawal rate indicates less mechanical allodynia. OP-AP group is analyzed against OP-saline group (*) and OP-NSAID group (#). * or #: P<0.05, * * or ##: P<0.01, * * * or ###: P<0.005.

FIG. 5 shows the GFAP immunoreactivity in spinal cord astrocytes 7 days after surgery.

FIG. 6 is a histogram showing the analyzed level of GFAP immunoreactivity shown in FIG. 5.

### DESCRIPTION OF THE PREFERRED EMBODIMENTS

The present invention provides a method of treatment of a neuropathic pain by administering an andrographolide to the subject. It is herein confirmed that andrographolide compounds can reduce the activity of astrocytes to relief the neuropathic pain induced by the central nervous system.

The causes of neuropathic pain induced by the central nervous system include a spinal cord injury (such as an interruption or dysfunction of pain transmission in the central nervous system), a cerebrovascular disease, multiple sclerosis, or a tumor. The causes of neuropathic pain induced by the peripheral nervous system include diabetic peripheral neuropathy, herpes zoster infection, HIV-AIDS, a chronic injury (such as from repetitive motion disorder), an acute injury (such as a surgical wound) or an autoimmune disorder. Although neuropathic pain can be caused by peripheral or central nervous, persistent peripheral nervous inflammation may also induce central nervous inflammation, and this may lead to an aggravation of allodynia, the symptom of sensing pain from normally non-painful stimuli.

A medicament containing andrographolide may include an acceptable carrier, diluent or excipient, and may be administered to a patient as oral medication, an intraperitoneal injection, intravenous injection, intramuscular injection or subcutaneous injection. The dose or frequency may be adjusted according to the level of pain.

The following embodiment is provided to illustrate the effects that an andrographolide compound has in alleviating neuropathic pain.

Preparation and Processing of the Medicament

Lyophilized andrographolide powder is dissolved in DMSO and subsequently diluted to 2.86 mM (1 mg/mL) in 90% normal saline and 10% DMSO for storage. The mixture is injected into the peritoneum (body cavity) of each animal (intraperitoneal injection) based on 5 mg/kg animal weight, for example, a 30 g mouse is injected with 150 μL of saline containing 0.15 mg of andrographolide. NSAID drugs (Diclofenac sodium salt) may be purchased from Sigma (D6899), and diluted to 2.86 mM (0.91 mg/mL) in 90% saline and 10% DMSO for storage. The NSAID drugs is administered via intraperitoneal injection based on 5 mg/ml animal weight, for example, a 30 g mouse is injected with 165 μL of saline containing 0.15 mg of NSAID drugs. The number of molecules administered is less in andrographolide treated mouse than in NSAID drugs treated rats. These drugs are administered daily after surgery.

The sciatic nerve transaction (SNT) is used for simulating a neuropathic pain model

An operation for inducing the sciatic nerve transaction of BALB/c mice is processing as follows. After isoflurane anesthesia is applied to male BALB/c mice, which have about 8 weeks of age and have a weight of approximately 30 g, the skin of the right thigh is slit to expose the muscle. Then a blunt instrument is used to spread open the biceps femoris to expose the sciatic nerve and its three nerve branches, which are the sural nerve, common peroneal nerve and tibial nerve. Then a sterilized no. 4-0 chromium wire is used to tie the sciatic nerve, and approximately 2-4 mm of the ends of the common peroneal nerve and the tibial nerve are cut at a position near the tying position. Care is taken to avoid touching and stretching the sural nerve. The muscle and skin are closed in two separate layers. For sham surgery, the sciatic nerve is exposed as described above but no contact is made with the nerve. In this embodiment, the nerve is tied to a level such that there is slight sheathing of the nerve in order to avoid affecting the blood circulation in the area. The mice's behavior is observed after several days of rest.

Manner of Administration

The experiment is separated into the following groups that receive different treatments. In the normal group, no surgery is carried out and no andrographolide or NSAID drugs are administered to mice. In the pain group (OP-saline), saline is given to mice daily after surgery. In the control group (Op-NSAIDs), Diclofenac sodium salt is administered to mice daily after surgery. In the experimental group (Op-AP), an andrographolide compound is administered to mice daily after surgery. The means of administration is by intraperitoneal injection. The concentration of drugs for both the control group and the experimental group is 5 mg/kg animal weight, and the average animal weight is 30 g. For the model that uses an in vivo sciatic nerve transaction to simulate pathological pain, the mice behavior is tested on 3, 7, 14 days after surgery. The von Frey hair test is used to determinate the withdrawal threshold of the pathological pain.

Behavior Test by Von Frey Test

Mechanical allodynia threshold at the lateral plantar surface of the hindpaw is assessed before nerve injury (as basal pain threshold) and then testing began on day 3 after surgery and continued for 7 and 14 days after surgery. Mechanical sensitivity is determined by measuring the withdrawal thresholds and reaction ratio to von Frey hairs (Stoelting, Wood Dale, Ill.) using the up and- down method (Dixon 1980) as described by Chaplan (Chaplan, Bach et al. 1994). Animals were placed in a plastic cage with a wire net floor and were allowed to habituate 10-15 min before testing began. Animals were also habituated over a period of 2-3 consecutive days by recording a series of baseline measurements. The filaments were applied in ascending order, each 5 times at an interval of 2-3 s to the plantar surface of the hindpaw (Bourquin, Suveges et al. 2006) and the smallest filament eliciting a foot withdrawal response is considered the threshold stimulus. The withdrawal threshold (force of the von Frey hair to which an animal reacts in over 50% of the presentations) is recorded. For measuring withdrawal rate, 0.02 g hair and 0.07 g hair is selected. For each ten tests when using specific hair, the percentage of withdrawal behavior exhibited is noted. Of the 4 documented behavior types, reaction type I is regarded as no behavior, and type II-IV is regarded as reactive which describing below.

I. No feeling (The mouse′ hindpaw does not withdraw after more than 3 seconds).

II. Lifting leg (The mouse lifts its leg slowly back to the original position about 1-2 seconds after touching and flinching from the von Frey hair.

III. Moving away (The detecting leg is moved and its position is switched, and the withdrawal takes approximately 1 second).

IV. Moving away quickly (The mouse's whole body is moved and its position is switched).

Statistical Analysis Method

The data measured in this experiment is expressed in terms of the mean±standard deviation, and the data from each experimental group is compared using One-Way ANOVA to find any significant difference between them. When P<0.05 indicates that there is a statistically significant difference. For convenience in the statistical analysis, the number of animals of each group in this experiment is 6 to 8.

Collection of Tissue Samples

The following procedure is to ascertain the activity of astrocytic glial cells of the spinal cord. The mice are deeply anaesthetized with sevoflurane and perfused transcardially with saline followed by 0.1 M phosphate buffer solution containing 4% paraformaldehyde. The L4-L6 segment of spinal cord and left and right sciatic nerves are dissected out and post-fixed in a 4% parformaldehye solution at 4° C. overnight, and transferred to a series of solution of phosphate-buffered saline (PBS), graded ethanol/PBS, xylyne, graded xylyne/wax mixture, and finally embedded in wax. A rotary slicer for tissues is used to slice the sample to a transverse section with a thickness of 3 um, and warm water is used to spread and attach to the glass slice.

Immunohistochemical Staining

The wax sections are dewaxed in a series of xylene, graded xylene/ethanol mixture, or graded ethanol mixture, and finally incubation in PBS. The antigen is recovered by an antigen retrieval solution (R&D Systems, Inc., 614 McKinley Place Nebr., Minneapolis, Minn. 55413). The sections of spinal cord are incubated with 0.3% H₂O₂ for 30 minutes to remove endogenous hydrogen peroxide. An ABC kit (Vector laboratory) is used for chromogen development; or instead no primary antibodies are used, but rather only a secondary antibody is used to mark the background. The sections is left to incubate with primary antibody at 4° C. overnight, wherein the primary antibody is glial fibrillary acidic protein (GFAP) (1:1000, Dako, Carpinteria, Calif. USA), and the secondary antibody for fluorescent staining is IgG conjugated to Alexa 488, (1:800, Molecular Probes, Eugene, Oreg.).

Quantification by Immunoreactivity (IR)

The immunoreactivity in each group is analyzed, and the intensity of chromogen staining or fluorescence is determined by photo form 4-5 independent animals. The image of the photo is analyzed using the same settings for quantitative comparison. All measured pixels in a specific area are analyzed by the ImageJ software on 0 (black) to 255 (white) scales. The total number of pixels having a value less than 50 in the photo is calculated and used to estimate the value of immunoreactivity in the area of interest. The data is analyzed using One-Way ANOVA, and P<0.05 (*) indicates that the analysis have a statistically significant difference.

Result of the Analgesic Effect of Andrographolide Compound on Neuropathic Pain

Sciatic nerve transaction surgery is performed to the male BALB/c mice to induce neuropathic pain, and a von Frey hair is used to apply different pressures of stimulation to the animal at 3, 7 and 14 days after surgery, and used to determinate withdrawal thresholds and withdrawal rate of the animals.

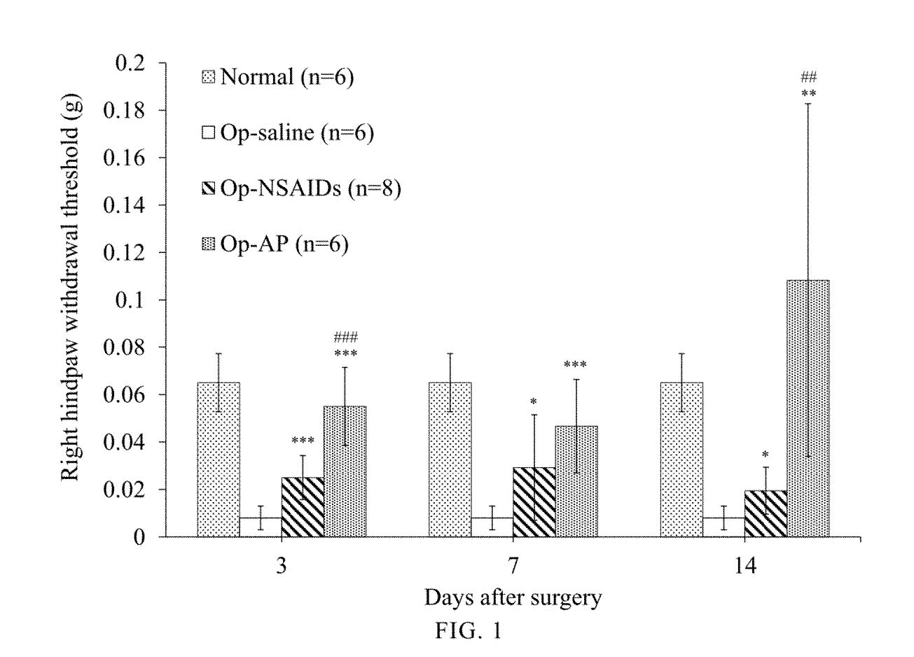

Please refer to FIG. 1, a histogram showing the mice's right hindpaw withdrawal threshold, the withdrawal threshold of the mice in the Op-AP group has a significant increase at 3, 7 and 14 days after surgery. In other words, the andrographolide compound effectively improves the allodynia behavior of the neuropathic pain of an animal when injected at a dose of 5 mg/kg animal weight. In the test, the grams of the NSAID drugs and the andrographolide given to the animal are equal, but the molecular weight of the andrographolide is greater than the molecular weight of the NSAID drugs, so that the number of moles of the NSAID drugs given to the animal is larger. The andrographolide compound has a more significant effect on the withdrawal threshold at the 3 and 14 days after surgery than the commercial NSAID drugs (The statistical p values are sequentially * or #: P<0.05, * * or ##: P<0.01, * * * or ###: P<0.005).

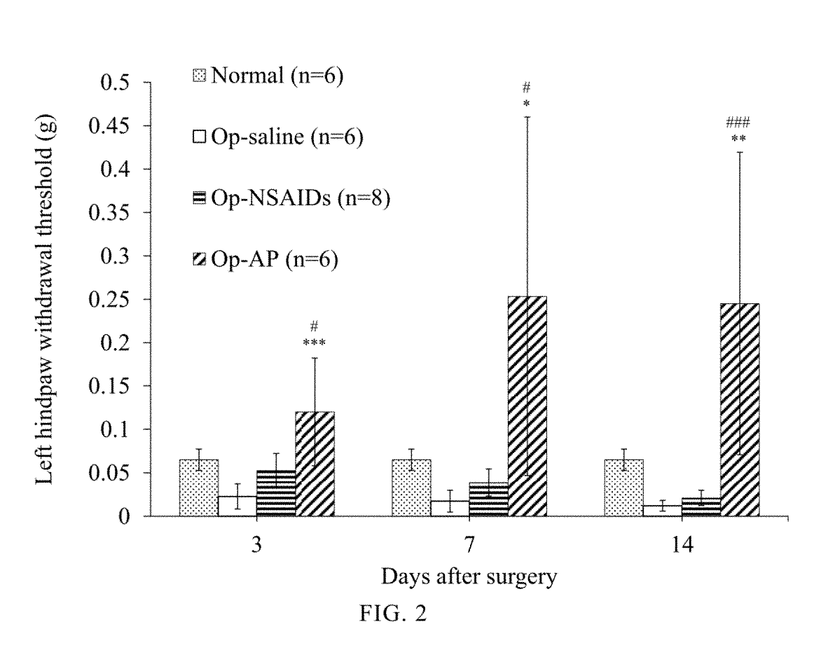

Please refer to FIG. 2, a histogram showing the left hindpaw withdrawal threshold, (where the left thigh has not received the sciatic nerve transaction surgery), since the sciatic nerve that has the problem is on the right side, and thus it is expected that the opposite side (left leg) will not have any reaction. However, nervous injury may cause central sensitization, and the sensation may extend from the right side to the left side in the spinal cord (the mirror pain). Thus, the behavior test of the nerves on contralateral hindpaw may also be assessing.

FIG. 2 shows that the withdrawal threshold of the mice in the Op-saline group is reduced at the 3, 7 and 14 days after surgery compared to the normal group. This demonstrated the mirror pain caused by allodynia in the contralateral side. At short time after surgery (3 days) NSAIDS provide certain degree of alleviation of the pain sensation, which is demonstrated by higher (although not significant) threshold compared to the saline group. This higher threshold does not sustain in 7-14 days post-operation. The withdrawal threshold of the Op-AP group is increased compared to that of the saline, and of the NSAIDs group in all 3 dates tested (* or #: P<0.05, * * or ##: P<0.01, * * * or ###: P<0.005).

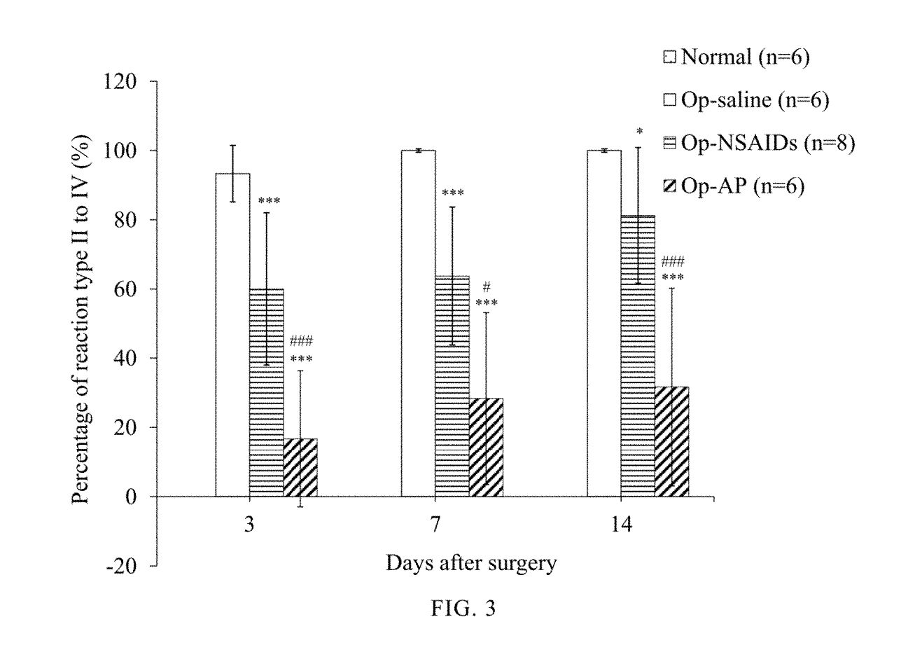

Please refer to FIG. 3, a histogram showing the withdrawal rate of a von Frey fiber of 0.02 g of the right hindpaw. The withdrawal rate is the value of effective occurrences of the reaction types (II˜IV) among all types of reaction (I˜IV). A lower withdrawal rate indicates a lower mechanical allodynia.

The result shows that the mice of the Op-AP group have a very significant low withdrawal rate at the 3, 7 and 14 days after surgery when compares with the Op-saline group. The OP-AP group has a very significant lower withdrawal rate at the 3, 7 and 14 days after surgery when compared with the Op-NSAID group, (the statistical p values are sequentially * or #: P<0.05, * * or ##: P<0.01, * * * or ###: P<0.005)), indicating a reduced pain behavior with use of andrographolide than the NSAID.

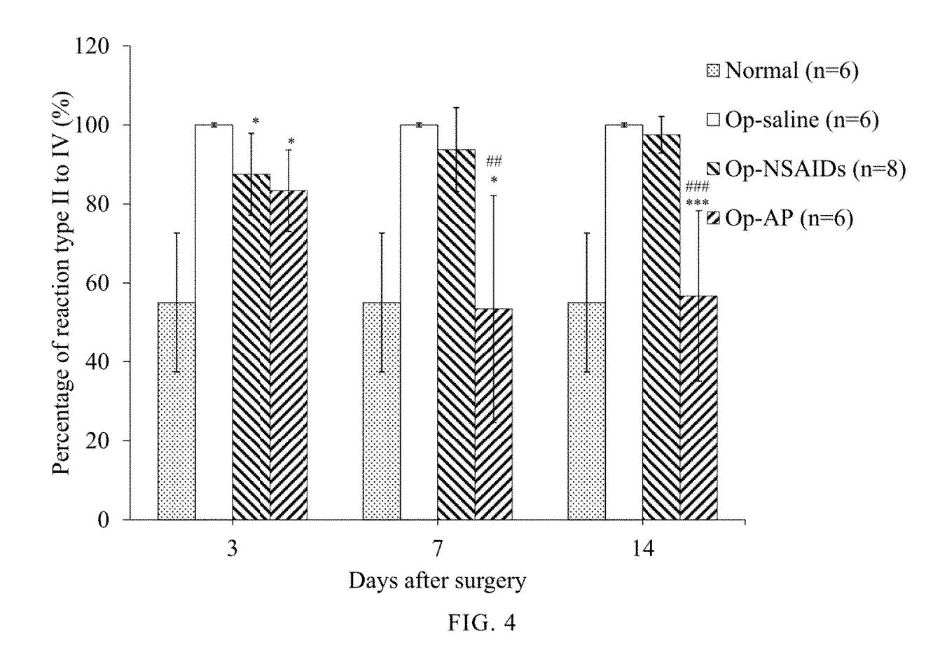

Please refer to FIG. 4, a histogram showing withdrawal rates when a fiber of 0.07 g is used. The result shows similar to when lighter fiber is used, the Op-AP group demonstrated reduced pain sensation compared to those not medicated (Op-saline group). At this fiber the difference between the Op-Ap and the Op-NSAID group is not significant at shorter time (3 days), but with the time the difference become significant (7-14 days)

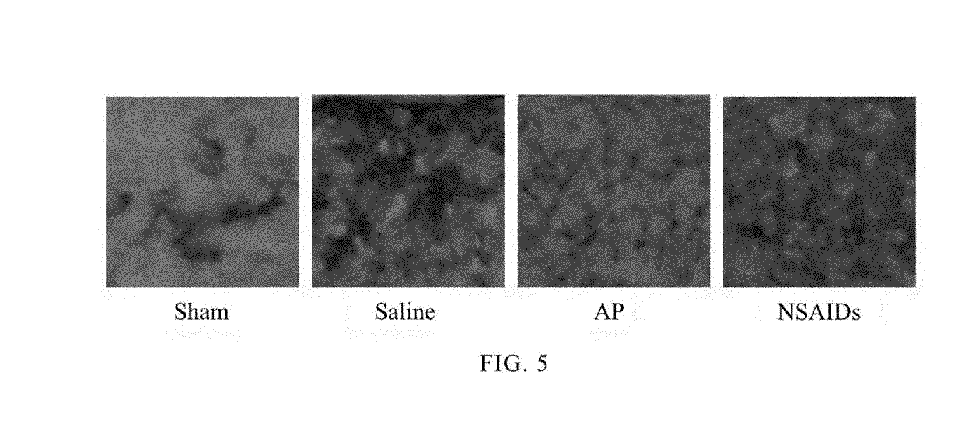

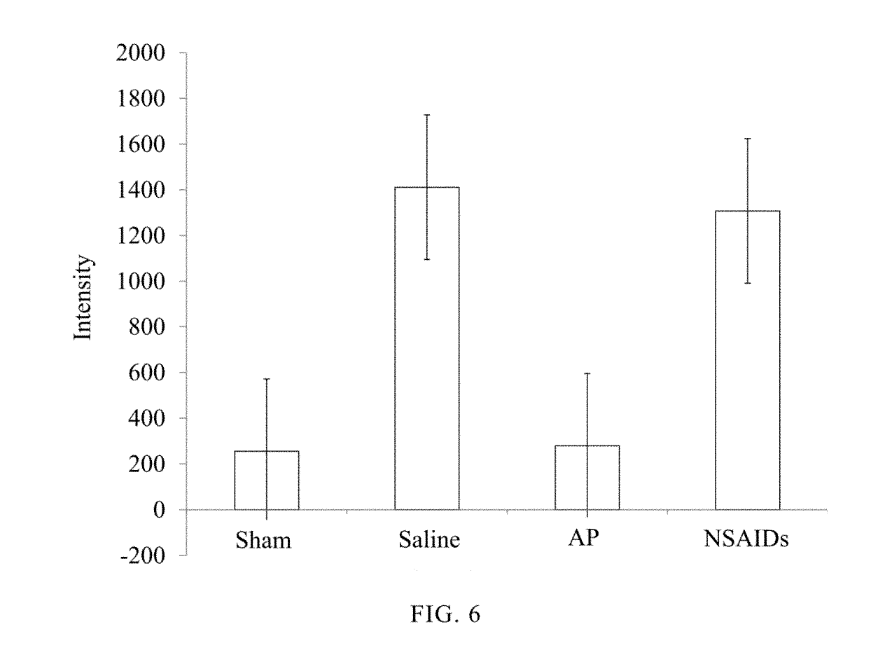

Please refer to FIGS. 5 and 6, which show the immunostaining of GFAP and quantification of astrocytic reaction at the 7 day after surgery. The behavior is consistent with that GFAP immunoreactivity. At this point in time, the inflammatory reaction in the spinal cord is already underway. The immunoreactivity of GFAP in L4-L6 spinal area is used to estimate the astrocytic reaction.

The results indicate the GFAP immunoreactivity of the astrocytes in the OP-AP group demonstrated a reduced trend toward to the OP-saline group. The results show that the andrographolide compound may contribute to the inhibition of the astrocytic activity in the spinal cord.

As disclosed in published literature, an upregulation expression of GFAP is generally regarded as an indicator of the astrocytic activation. The upregulation expression of GFAP and astrocytic hypertrophy are related to pains found in many pathological damages. As shown in FIGS. 1 and 2, the mice feel pain at both the right hindpaw and left hindpaw, (and these are mirror pains). As to the astrocytes with lots of gap junctions, the inhibition of the gap junction has an analgesic effect, particularly concerning the transmission of mirror pains. The astrocytic reaction lasts longer than the microglial reaction (up to 150 days), and the activity of the astrocytic reaction occurs independently from the activity of the microglial reaction. The activated astrocytes are sufficient to cause pain.

As described above, the results show that the andrographolide compound can provide relief from mechanical allodynia and allergic reactions, and the degree of relief is better than using the same dose of the NSAID drugs. The reduction in mechanical allodynia is consistent with the lower activity of astrocytes.

While the present invention has been described by means of a specific embodiment, numerous modifications and variations could be made thereto by those skilled in the art without departing from the scope and spirit of the invention set forth in the claims.

What is claimed is:

1. A method of treatment of a neuropathic pain in a subject, comprising:
administering an andrographolide to the subject to relief the symptom of the neuropathic pain by reducing the activity of astrocytes.

2. The method of claim 1, wherein the reducing of the astrocytic activity includes reducing the cellular level of glial fibrillary acidic protein (GFAP).

3. The method of claim 1, wherein the neuropathic pain is a central nervous system pain.

4. The method of claim 1, wherein the causes of the neuropathic pain include spinal cord trauma, cerebrovascular disease, multiple sclerosis, a tumor, diabetic peripheral neuropathy, herpes zoster, a HIV-AIDS, a chronic injury, an acute injury or an autoimmune disorder.

5. The method of claim 1, wherein the andrographolide is administered to the subject in a therapeutically effective amount.

6. The method of claim 1, wherein the andrographolide is administered orally, intraperitoneally, intravenously, intramuscularly, or subcutaneously to the subject.

7. The method of claim 1, wherein a medicament containing the andrographolide includes an acceptable carrier, a diluent, or an excipient.
